(19) United States
(12) Patent Application Publication
(10) Pub. No.: US 2010/0074924 A1
(43) Pub. Date: Mar. 25, 2010

(54) MEMBRANE PROTEIN SM29 OF SCHISTOSOMA MANSONI AND USES THEREOF FOR TREATING AND DIAGNOSING SCHISTOSOMIASIS

(76) Inventors: Sergio Costa Oliveira, Belo Horizonte (BR); Fernanda Caldas, Belo Horizonte (BR)

Correspondence Address: NIXON & VANDERHYE, PC, 901 NORTH GLEBE ROAD, 11TH FLOOR, ARLINGTON, VA, 22203, US

(21) Appl. No.: 12/297,622

(22) PCT Filed: Apr. 17, 2007
(86) PCT No.: PCT/BR2007/000094

§ 371 Date: Sep. 15, 2009

(30) Foreign Application Priority Data
Apr. 17, 2006 (BR) PI0604176-0

(51) Int. Cl. A61K 39/002; C07K 14/00; C12P 21/00; G01N 33/53; A61P 33/12

(52) U.S. Cl. 424/265.1; 530/350; 435/69.1; 435/7.1

(57) ABSTRACT

The present invention relates to a Sm29 membrane protein and to an immunoenzymatic assay (diagnosis) capable of detecting specific IgG antibodies against the Sm29 protein present in the serum of schistosomiasis patients, the use of Sm29 as a vaccine in the prevention of schistosomiasis and the use of Sm29 in the treatment of allergic diseases. The immunoenzymatic assay is capable of detecting specific IgG antibodies against Sm29 present in the sera of individuals with schistosomiasis. Support-adsorbed Sm29 is reacted with the test sera. After incubation with the conjugate, the reaction is developed with a solution composed of the enzyme substrate used in the conjugate (chro-mogen). After the development of the reaction, it is paralyzed with the acid solution and read in a spectrophotometer. The vaccine using Sm29 is capable of reducing the number of adult worms in vaccinated animals in 31.2% (without adjuvant); 51% and 56.7% (with adjuvant). Vaccination is also capable of reducing the number of parasite eggs in the intestine in 37.6% and 60%, and the number of granulomas in the liver of the host in 48% and 61%. Vaccination is done by using 10-50 μg of recombinant Sm29 with or without the use of a sub-cutaneously-applied adjuvant. After 15 days, two booster doses are applied with a 15-day interval containing 10-50 μg of recombinant Sm29 with or without the use of an adjuvant. The stimulation of mononuclear cells of the peripheral blood of asthmatic patients shows that Sm29 is capable of inducing a high production of IL-10 the innate immune system cells, thus evidencing its potential use as a therapeutic in the treatment of allergic diseases.

The present invention refers to the production of recombinant proteins by means of genetic engineering techniques (recombinant DNA), specifically the Sm29 protein.

The present invention also relates to the process for obtaining a vaccine vehicle, a vaccine comprising Sm29 and pharmacologically and physiologically carriers and applications of that pharmaceutical composition.

Finally, the present invention discloses the obtainment of an immunoenzymatic assay for detecting schistosomiasis.

Schistosomiasis is a chronic endemic disease affecting more than 200 million people, and other 600 million people are under the risk of infection in 76 countries in the world (Ross, 2001). The major agents causing schistosomiasis are Schistosoma mansoni, Schistosoma japonicum and Schistosoma haematobium. In Brazil, it is estimated that there are approximately 12 million people infected by S. mansoni (Kloetzel, 1989). The main states affected are Minas Gerais and Bahia, but the infection extends to the states of Maranhão and Pará.

Schistosoma mansoni is transmitted to humans through water containing contaminated snails of the genus Biomphalaria. Upon transmission, the parasite infects man by penetrating the skin in the form of cercaria, then transforming itself into a schistosomulum. It migrates to the lungs and then to the liver, where sexual maturation is concluded and male and female adult worms copulate. Egg laying in the liver followed by their retention in the human tissue is the main cause of the severe pathologies associated with this disease (Gryseels et al., 2006). At the moment of transmission, an allergic reaction may occur in the skin caused by the penetration of the parasite. Fever, headache, chills, sweating, weakness, lack of appetite, muscular pain, cough and diarrhea are the symptoms of schistosomiasis in its acute phase. The liver and the spleen also increase in size due to inflammation caused by the presence of the worm and its eggs. If untreated, the disease may evolve into its chronic form, in which the feces generally are accompanied by blood. The infected individual experiences dizziness, palpitation, impotence, weight loss and a markedly enlarged liver. Some symptoms result from the obstruction of spleen and liver veins, and the blood flow deviation can even cause varices in the esophagus. In this stage, the appearance of the infected individual is characteristic: weak, but with an enlarged belly, which is why the popular name for the disease in Brazil is “water belly.”

The strategies currently used to control schistosomiasis consist of transmission and morbidity control. Transmission control intends to interrupt the evolving life-cycle of the parasite, preventing the infection of new cases. In morbidity control, the objective is to prevent the appearance of hepatosplenic forms. This objective is achieved through the diagnosis and chemical treatment of patients using praziquantel and oxaminiquine. However, these strategies have limitations, the major one being the development of resistance to the drugs used in the treatment (Ismail et al., 1999; Stelma et al., 1995). Bergquist et al. (1998) have shown that, in the last decades of chemical treatment against schistosomiasis, the number of people infected has continued the same, clearly indicating the need to develop an antischistosomal vaccine.

U.S. Pat. No. 5,597,570 relates to the development of a vaccine against schistosomiasis. It relates to a protein which includes the epitopes of the p28 protein, a poxvirus containing a gene coding for said protein, a cell incorporating a vector for the expression of said protein, a method for preparing said protein, a DNA sequence coding for the p28 protein, a pharmaceutical composition, antibodies raised against said protein and their application by way of diagnostic agents for schistosomiasis.

U.S. Pat. No. 5,730,984 discloses a vaccine against helminth infection, comprising a Sm14 protein of S. mansoni. The invention relates to a helminthic derived antigenic material capable of inducing effective and long lasting protection against parasites, in particular to-antigens that mediate protective immunity against helminths. Furthermore, an immunogenic composition capable of providing partial protection against pathogenic helminths is also disclosed, comprising an effective addition of the isolated Sm14 protein, protecting against S. mansoni.

U.S. Pat. No. 5,804,200 relates to the obtainment and use of proteins and immunogenic parasitic nematode vaccines derived from proteins isolatable from the L3 and L4 larval stages of nematodes parasitic in mammals, and including a protein of about 20.5 kDa. The proteins of that invention are identified with the use of biological materials used to destroy or impair the parasitic nematode in a host.

U.S. Pat. No. 6,372,219 relates to a protein with anti-inflammatory and immunomodulatory functions, identified and isolated from the secretions of a human parasite Schistosoma mansoni. This protein, designated as Sm16.8, is released by schistosomes upon entry into human skin.

Since the seventies, several studies have been carried out with a view to assess the immunogenicity and complexity of the S. mansoni tegument. The adult worm tegument consists of a cytoplasmatic syncytium covered by a single double layered membrane (MacLaren & Hockley, 1977). Membrane antigens and surface-associated antigens present in the S. mansoni tegument have great potential in the development of vaccines for being exposed to the immune system of the host, and they are also very important in the biological studies of the parasite-host interface.

The first studies related to the immunogenicity of the S. mansoni tegument were based on the direct vaccination of animals with the tegument of the purified parasite and western blot analyses (Hackett et al., 1985; Payares et al., 1995; Simpson et al., 1985; Simpson et al; 1986; Smithers et al., 1989; Smithers et al., 1990). These studies revealed some important antigens recognized by the serum of vaccinated animals and patients with the chronic form of the disease.

With the advancement of molecular biology technologies, several antigens present in the S. mansoni tegument have been characterized. GAPDH (Goudot-Crozel at al., 1989), Sm25 (Ali et al., 1991), Sm22 (Jeffs et al., 1991), paramyosin (Laclette et al., 1991), Sm15 (Abath et al., 1993, 1994), Sm23 (Lee et al., 1995), actin (Oliveira et al., 1995), Sm10 (Kohlstadt et al., 1997), Sm13 (Abath et al.; 2000), Sm16 (Rao et al., 2002), Sm8 (Abath et al., 2002), Sm-TSP-1, Sm-TSP-2 and other less characterized proteins (Smyth et al., 2003) are described in the literature. It should be noted that several of these proteins mentioned lack characteristics of membrane-associated proteins (lack of transmembrane helices and lipid anchor sites).

The immunological properties of some membrane proteins of S. mansoni have been investigated since the last decade. Koster et al. (1993) characterized the immunoreactivity of human sera against the surface antigen Sm23, showing that anti-Sm23 antibody titers varied widely in infected patients. Sm13 was investigated by Abath et al. (2000), and the presence of anti-Sm13 antibodies was found in the sera of schistosomiasis patients. Recombinant Sm25 was tested in mouse vaccination trials by Suri et al. (1997), producing high antibody titers, but protection against subsequent cercaria challenge was not observed.

Scientific advances involving the genome and transcriptome projects have provided important information in the identification of antigens for the development of vaccines, the diagnosis and understanding of the parasite-host relationship in several diseases. The S. mansoni transcriptome, characterized by the Verjovisk-Almeida et al. group (2003) and by the Minas Gerais Genome Network (“Rede Genoma de Minas Gerais”), is an example. As verified by these groups, vaccine candidates preferably include surface-exposed proteins, expressed in mammal-infecting parasite stages. Proteins orthologous to toxins, host factor receptors, molecules involved in cellular adherence, membrane proteins or surface-exposed proteins and enzymes are considered to be potential candidates for the development of vaccines.

Studies performed to develop the present technology have led to the isolation and identification of a candidate protein for the development of a diagnosis and a vaccine against schistosomiasis, called Sm29. This selection was made based on analyses of genome sequences annotated with bioinformatic tools with access to the following databases: GenBank, dbEST (data base Expressed Sequences Tags), KOG (Eukaryotic Orthologous Groups) and UNI-PROT (Universal Protein Resource)/GOA (Gene Onthology Annotation) as the source of information.

The empirical identification of the typical regions or domains of secreted or cytosolic membrane proteins is very important for a more detailed study of the actual structure and functions of the selected antigens. In the case of membrane proteins, the presence of the signal peptide, GPI anchor sites, transmembrane region and glycosylation sites are very important characteristics for their identification. The search for conserved domains in these proteins is also capable of revealing characteristics for determining the biological function and importance of these selected antigens.

Analyses of the primary amino acid structure of Sm29 were carried out using ExPASy (Expert Protein Analysis System) (Gasteiger et al., 2003), which is a free access, public site of bioinformatic software. The presence of a signal peptide (export sequence to extracellular media) was identified through the SignaIP 3.0 software (Bendtsen et al., 2004; Nielsen et al., 1997). The presence of transmembrane helices and GPI (glycosylphosphatidylinositol) anchor sites were analyzed using the TMHMM and DGPI software (Kronegg et al., 1999), in this order. Potential O-glycosylation and N-glycosylation sites have been identified through YinOYang (www.cbs.dtu.dk/services/YinOYang) and NetNGlyc 1.0 (http://www.cbs.dtu.dk/services/NetNGlyc/), respectively. Finally, SYFPEITHI (Rammensee et al., 1999) was used in predicting human MHC (Major Histocompatibility Complex) or HLA (Human Leukocyte Antigen) class II binding epitopes.

Antigen Sm29 presents an N-terminal signal peptide having 26 amino acids (1 MKSGWEYIGIFLYIMVNILDKQRCHS 26), 3 potential O-glycosylation sites (T39, T132 and T133), 2 potential N-glycosylation sites (N58 and N115) and one primary C-terminal transmembrane helix (LSIHRHVIIVLFVCIGISKYIL). In addition, it has several HLA binding peptides (HLA DRB1*0101, HLA-DRB1*0301 (DR17), HLA-DRB1*0401 (DR4Dw4), HLA-DRB1*0701, HLA-DRB1*1101 and HLA-DRB1*1501 (DR2b) (Table I), showing that this antigen is capable of binding to receptors involved in immune response activation and inducing an effector response in combating schistosomiasis, involving the production of antibodies and the activation of T lymphocytes.

TABLE I
High affinity human MCH II binding peptides with
increased promiscuity between the HLAs analyzed.
The anchor amino acids are in bold.

| HUMAN MHCII | | |
|---|---|---|
| (HLA-DRB1) | Position | Peptide |
| 0101-0401-1101 | 5 | VCDYCPIVTSVSISE |
| 0101-0401-1101 | 82 | FRCYTCLNCTKSNQK |
| 0101-0301-0701 | 38 | ICVFKDGIPINFPNE |
| 0101-0301-0401- | 62 | CNGLTVDNTGKIPSV |
| 0701 | | |
| 0301-1101-1501 | 64 | GLTVDNTGKIPSVPI |

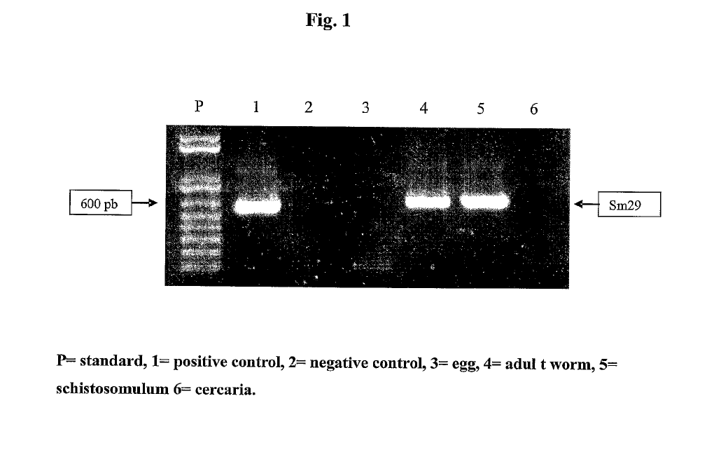

The Sm29 transcription analyses in the parasite stages show that it is expressed in the adult worm phase and in the lung-stage schistosomulum as shown in FIG. 1. FIG. 1 refers to the analysis of the Sm29 transcription in the S. mansoni human infecting phases through PCR using specific initiators for Sm29 and cDNA libraries of the analyzed phases. Thus, it is inferred that this antigen is capable of combating schistosomiasis in recent phases of the infection, caused by the schistosomulum, not enabling the development of more severe forms of the disease.

The helminths comprise approximately 100 families. Most are comparatively harmful parasites, living in the intestine and in other organs of vertebrates. The trematodes that cause serious problems in humans are the schistosomas or trematode sp, and the parasites which more commonly infect animals are the lung and liver trematodes.

Fasciola is the most important of these liver trematodes, infecting mainly domestic ruminants, responding for high economic losses all over the world (bovine, caprine and ovine animals).

The major characteristic of this disease, which is responsible for the pathology, morbidity and mortality of the abovementioned animals, is the destruction of the host's liver tissues and damage to the bile ducts. Morbidity is extremely high in young animals. Fasciola may also parasitize man, when there is the opportunity, and is more frequent in Cuba and in Latin-American countries. However, the actual trematode that infects the human liver is Clonorchis sinensis, very common in China, Japan, Korea, Vietnam and India. Pathology is basically caused by the enlargement of the bile duct walls and may cause cirrhosis and death.

Both Fasciola and Clonorchis have passive entry as metacercarias ingested with food (vegetables and fish for fasciola and clonorchis, respectively), but their migration routes to the bile duct in the vertebrate body differ. The present invention discloses the use of an antigen with protective action against helminth infection in humans and animals and to an method of immunoprophylaxis of helminthological diseases of interest to human and veterinary medicine.

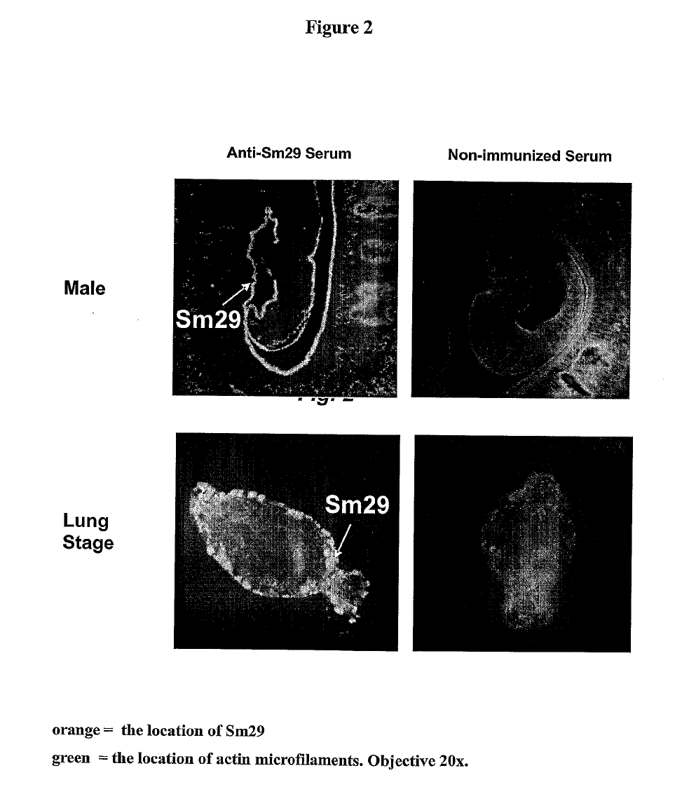

Immunolocation assays using sera from experimental animals vaccinated with recombinant Sm29 show that this protein is present in the adult worm tegument and in the lung-stage schistosomulum of S. mansoni. FIG. 2 refers to immunolocation of Sm29 in the adult male worm and in the lung-stage schistosomulum of S. mansoni. Therefore, the immune system can easily recognize this antigen and trigger an effector immune response in combating the parasite, eliminating adult worms and reducing the possibilities of egg reproduction and release.

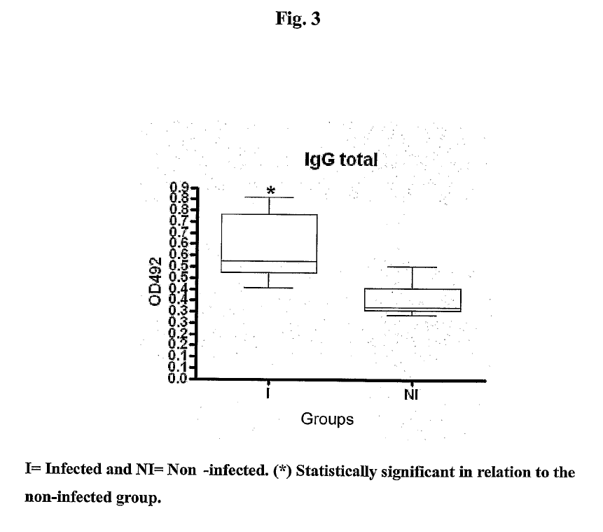

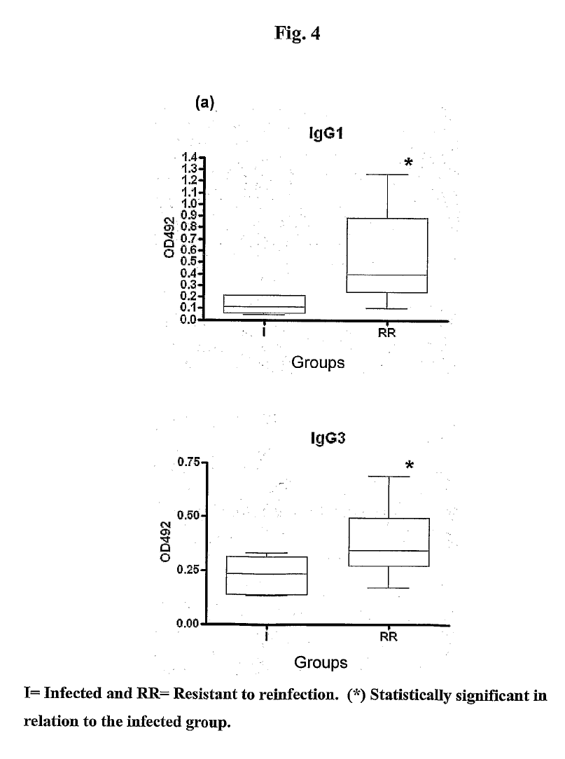

Studies in patients residing in endemic areas for schistosomiasis have shown promising results for the development of a diagnosis test and a vaccine against schistosomiasis. Using the ELISA technique, a high production of Sm29r-specific IgG isotype antibodies is found in schistosomiasis patients (diagnosed by 3 Kato-Katz exams) compared with healthy individuals with no history of the disease, which enables to differentiate between sick and healthy individuals as demonstrated in FIG. 3 which shows a profile of total specific anti-Sm29 IgG in patients in endemic areas for schistosomiasis compared to healthy individuals. It was also observed, through the ELISA technique, a high production of anti-Sm29 IgG1 and IgG3 isotype antibodies in schistosomiasis-resistant individuals (see FIG. 4 related to a profile of specific anti-Sm29 IgG1 (a) and IgG3 (b) in patients of endemic areas for schistosomiasis. This data show that specific antibodies against Sm29r are related to resistance against primary infection of individuals with a negative stool examination and resistance against reinfection of individuals with a negative stool examination after treatment with praziquantel, all dwellers of endemic areas and in frequent contact with contaminated water.

Another interesting aspect observed in schistosomiasis is immunoregulation, wherein schistosomiasis patients present immune system modulation. This is a strategy found in several parasitoses caused by helminths which reduces the pathology, or severity, of the disease and increases the longevity of the parasite in the host (Maizels & Yazdanbakhsh, 2003). Experiments assessing the immunoregulation observed in patients and experimental animals have revealed a high production of IL-10, the cytokine responsible for the immune response modulation. The suppression of Th2 immune response is directly involved in the reduction of allergies, and this fact has already been found in endemic areas for schistosomiasis, where patients show a reduction in the reactivity against allergens and present lighter forms of asthma (Araújo et al., 2006).

Allergic diseases and/or symptoms caused by contact with allergens are, in general, chronic and of palliative treatment. Most of the medicaments prescribed in connection with these diseases are directed to relieving the symptoms and do not have a curative effect. Other treatments, called substitution therapies, involve long periods of substance administration, which are needed due to reduced or insufficient production of said substances. The current treatments are mostly dissatisfactory, involving a series of side effects and a mere delay in preventing disease progression. Therefore, methods for treating and improving pharmaceutical compositions with application to allergens and allergic diseases are needed.

Inteleukin-10 (IL-10) was recently described as a natural, endogenous cytokine with immunosuppressive action. Interleukin IL-10 has a strong influence on the biological chemotaxis, proving to have potential regulatory effects on the in vivo and in vitro immunological responses. IL-10 also inhibits the chemotactic effect of any substance from a group of low molecular weight cytokines, capable of reducing leukocyte chemotaxis or chemokinesis in inflammations. This enables one to state that, the role of IL-10 having been acknowledged as a deactivator of macrophage functions and an inhibitor of Th2 activity, drugs capable of stimulating the production of IL-10 may have a therapeutic effect on diseases characterized by a deficiency in the production of this cytokine.

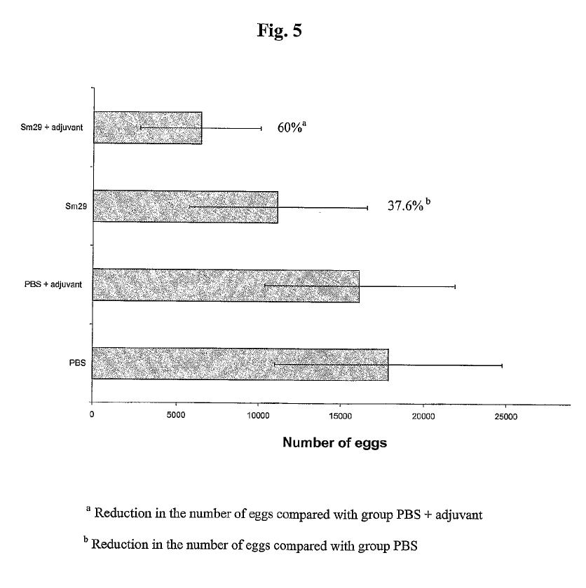

Data show that Sm29 is capable of stimulating the production of IL-10 by innate immune system cells, since asthmatic patients responded as well as asthmatic patients with schistosomiasis. In addition, mononuclear cells from the peripheral blood of asthmatic patients have shown a higher production of IL-10 when stimulated with allergen 1 of acarus D. pteronyssinus (Derp-1), a common agent causing respiratory allergy, together with the Sm29 protein, and compared with Derp-1 alone (see FIG. 5 related to the count of eggs in the intestine of mice vaccinated with recombinant Sm29 and challenged with 30 cercarias of S. mansoni. Count made 45 days after challenge). In accordance with this data, it was noted that Sm29 has the potential of being used in the treatment against allergens and allergic diseases.

To assess the occurrence of infection by Schistosoma mansoni, the present invention further relates to the production of a kit, consisting of a methodology for detecting specific IgG antibodies against Sm29 in the serum of symptomatic patients for schistosomiasis using the ELISA (Enzyme-Linked Inmunosorbent Assay) technique. The kit consists of a 96-wells microtitration plate or any other solid support such as tubes, spheres, nitrocellulose paper or nylon; blocking buffer with 10% fetal bovine serum or any other solution with proteins that do not interfere in the antigen-antibody reaction; carbonate/bicarbonate buffer, pH 9.6 or any other buffer or method enabling the adsorption of Sm29r in a solid support; recombinant, purified and desalinized Sm29; test serum and control serum diluted 1:50 in buffer-tween, any buffer at physiological pH being accepted for dilution; human peroxidase-conjugated anti-immunoglobulin or any other conjugate having specificity for human immunoglobulins and using other enzymes, such as acetylcholinesterase, lactate dehydrogenase, β-galactosidase, glucose oxidase and alkaline phosphatase; orthophenylenediamine substrate or any other chromogen substrate recognized by the enzyme used in the conjugate, sulfuric acid or any other solution that paralyses the reaction; spectrophotometer or any other instrument capable of measuring the intensity of the color formed.

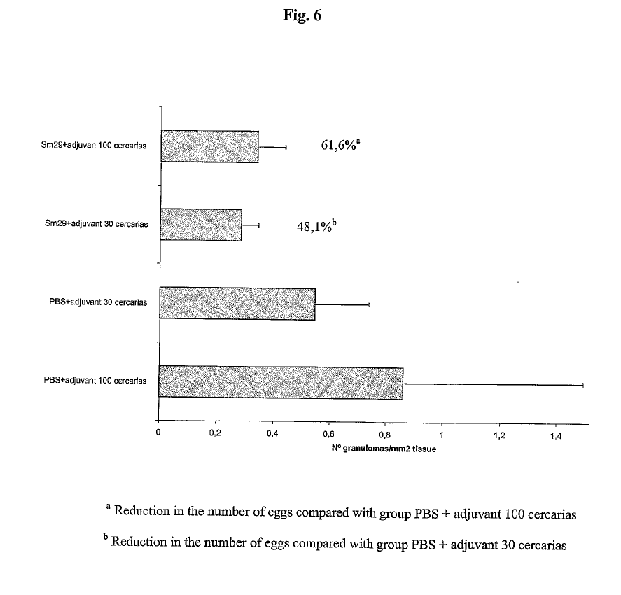

The immunoenzymatic assay is performed in 7 phases as shown in FIG. 6 which contains a graph of the count of granulomas in the liver of mice vaccinated with recombinant Sm29 and challenged with 30 or 100 cercarias of S. mansoni. Count made 45 days after challenge.

In phase 1, the 96-wells microtitration plates are adsorbed for 16 h at 4° C. with 100 μl of recombinant Sm29 at a concentration of 5 μg/ml in a 0.1 M carbonate bicarbonate buffer (pH 9.6) per well.

In phase 2, the supernatant is discarded and the plates are blocked with 10% fetal bovine serum in PBS (pH 7.4) for 2 h at ambient temperature. After blocking, in phase 3, the plates were washed 3× with PBS with 0.05% tween20 (PBST) and, after drying, the diluted sera (1:50) have been added in duplicate 100 μl per well and incubated for 1 h at ambient temperature. After incubation, in phase 4, the plates are washed 3× with PBST and 100 μl is added per well of peroxidase-conjugated anti-human IgG mouse antibody at the concentration of 1:10000 and incubated for 1 hour at 37° C.

Following incubation, in phase 5, the plates were washed and developed with orthophenylenediamine (OPD) with the addition of 0.05% hydrogen peroxide in a citrate 0.1M buffer, ph 5. The development is interrupted in phase 6, with 5% H₂SO₄ after 30 min of incubation. Test reading occurs in phase 7, at 492 nm.

Immunization method—Animal vaccination with Sm29r is done in three doses in a 15-day interval aiming at keeping a high production of specific antibodies and activating the cellular immune system, ensuring the formation of an immunological memory, which may be performed with any other amount of doses, provided they are sufficient to keep a high stimulation rate: 1^{st}. Vaccine containing 10-50 μg of the Sm29r protein with or without the use of Freund's adjuvant; 2^{nd}. Dose through subcutaneous injection; 3^{rd}. After 15 days, in the third phase, a first booster dose is effected with 10-50 μg; 4^{th}. After 30 days of the first dose, in phase four, the second booster dose is applied with 10-50 μg of Sm29r protein with or without the use of Freund's adjuvant.

Sm29r is usually administered subcutaneously but it may be administered by another other route capable of inducing the production of antibodies and a specific cell response for Sm29r. The methodology used for animal vaccination using Sm29r and Freund's complete and incomplete adjuvant is described in four phases.

The preparation of the vaccine for immunization presents a formulation containing 10-50 μg of the Sm29r protein with or without the use of Freund's adjuvant.

The Freund's adjuvant associated with Sm29r is used to potentiate the formation of humoral and cellular response, and this may be replaced with any other adjuvant capable of activating the formation of a satisfactory humoral and cellular immunological response. The present invention may also be understood by the non-limiting examples listed below:

### EXAMPLE 1

### Pre-Clinical Vaccination Assays in Experimental Animals

Clinical assays revealed the production of high levels of specific antibodies against Sm29 and a reduction in the number of adult worms of about 31.2% and 56.7% (with and without adjuvant, respectively) upon infection with 30 cercarias, and 51% (with adjuvant) upon infection with 100 cercarias in animals vaccinated with Sm29 compared with non-vaccinated animals (Table 2).

TABLE 2
Protective immunity in C57BL/6 mice induced by vaccination with
re-combinant Sm29 in addition to the Freund's complete
and incomplete adjuvant.

| Groups | Male worms | Female worms | Total worms | Protection |
|---|---|---|---|---|
| Infection | | | | |
| with 30 | | | | |
| cercarias^{a} | | | | |
| Control | 8.37 ± 1.8 | 7.5 ± 3.25 | 16 ± 3.96 | |
| PBS | | | | |
| Sm29r + | 6.8 ± 1.9 | 5 ± 1.51 | 11 ± 4.06 | 31.25% |
| PBS | | | | |
| Control | 7.92 ± 3.6 | 7 ± 2.7 | 15.28 ± 4.26 | |
| CFA/IFA | | | | |
| Sm29r + | 3.93 ± 2.1 | 3.05 ± 1.3 | 6.54 ± 2.45* | 56.7% |
| CFA/IFA | | | | |
| Infection | | | | |
| with 100 | | | | |
| cercarias | | | | |
| Control | 28.28 ± 8.1 | 29.42 ± 6.47 | 57.71 ± 12.21 | |
| CFA/IFA | | | | |
| Sm29r + | 14.62 ± 1.7 | 15.12 ± 4.7 | 29.75 ± 5.3* | 51% |
| CFA/IFA | | | | |

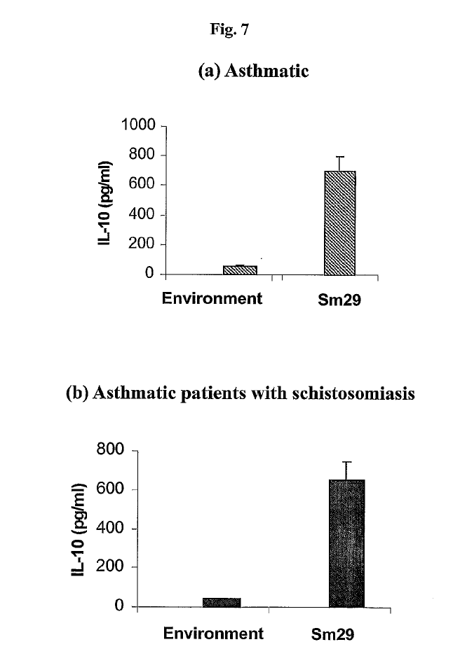

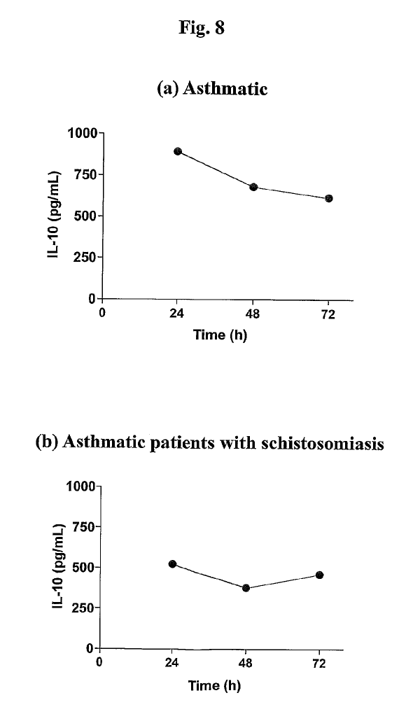

It is also noted that there is a reduction of 60% and 37.6% in the egg laying in vaccinated animals with Sm29 associated or not with the adjuvant, respectively, and infected with 30 cercarias (see FIG. 7 related to the assessment of IL-10 production in asthmatic patients (a), infected with Schistosoma mansoni and asthmatic patients (b) in the presence or absence of allergen Derp-1 (c). *P<0.05 compared with the environment) and a reduction in the number of granulomas present in the liver of about 48% and 61% in animals vaccinated with Sm29r associated with an adjuvant and infected with 30 and 100 cercarias, respectively (FIG. 8=Kinetic assessment of the IL-10 production in asthmatic patients (a) and asthmatic patients infected with Schistosoma mansoni (b)).

### EXAMPLE 2

### Assessment of the IL-10 Production in Infected Asthmatic Patients

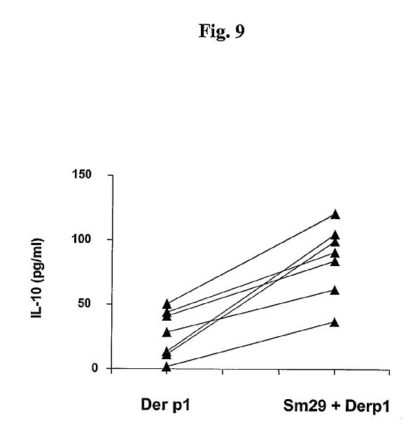

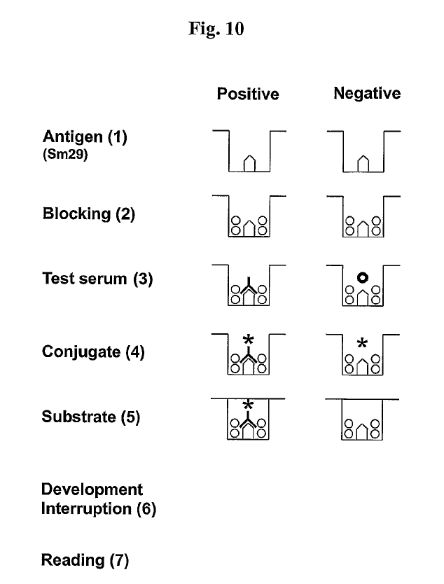

The assessment of IL-10 production in asthmatic patients and asthmatic patients infected with S. mansoni has shown that this antigen is capable of inducing a high production of this cytokine in both groups (FIG. 9). This data was obtained by means of assays with mononuclear cells of the patient's peripheral blood (CMSP). These cells were obtained through Ficoll-Hypaque gradient separation, and incubated at 37° C. at 5% CO₂ in a RPMI 1640 media, containing 10% of normal inactivated human AB⁺ serum, 100 U/mL penicillin, 100 μl/mL streptomycin, 2mM L-glutamine, 30 mM HEPES (GIBCO BRL, Gaithersburg, Md.) in 24-well plates. The cells were stimulated with 10 μg/ml of Sm29r during 6, 12, 24, 48 and 72 h. Polymixin B sulfate (30 μg/mL) was added to the cultures in order to block the production of cytokines by LPS contained in Sm29r. This contaminating LPS is derived from the E. coli bacteria used in the recombinant production of the S29 protein. The levels of IL-10 were determined by the sandwich ELISA technique using a commercially available kit (R&D Systems). A standard curve has been used to express the results in pg/mL. The production of IL-10 in these patients has been high during the entire culture incubation, the largest production being found in asthmatic patients only (891±213 pg/mL in 24 hours, 681±501 pg/mL in 48 hours and 618±191 pg/mL in 72 hours) (see FIG. 10 related to immunoenzymatic assay for schistosomiasis IgG-Sm29).

### EXAMPLE 3

### Production of the Recombinant Sm29 Protein

The recombinant protein was obtained in a prokaryote system, and any other prokaryote or eukaryote recombinant expression system may be used which is capable of producing the recombinant protein in question. To obtain the Sm29r protein, the cDNA fragment encoding the protein corresponding to the amino acid sequence region 40-169, without the N-terminal signal peptide and C-terminal transmembrane helix, is expressed in E. coli. This cDNA fragment has been subcloned in expression vector pET21 a (Novagen, Madison, Wis., USA), which expresses the recombinant protein with a C-terminal fusion of six histidines. For subcloning into an expression vector, a PCR (polymerase chain reaction) is performed, amplifying the desired Sm29 fragment, using specific initiators for the fragment ends. The subcloning into an expression vector and the confirmation of the DNA sequence are done according to Sambrook et al. (1989).

In order to induce the expression of Sm29, 1 liter of culture containing LB media and bacteria BL21(DE3) with the recombinant plasmid is grown at 37° C. until OD₆₀₀ of 0.5-0.8. After reaching the desired OD₆₀₀, the culture is induced with IPTG during 5 hours, the cells collected by centrifugation and resuspended in a lysis buffer. The cells are lysed by sonication and the recombinant Sm29 contained in the inclusion bodies is resuspended in a denaturating buffer.

The purification of recombinant Sm29 is done in an affinity system for a six-histidine tail. This methodology enables a large-scale production with the advantage of obtaining a highly purified material, eliminating undesirable contaminating proteins present in cell cultures. To this end, the purification of the recombinant protein is performed by nickel affinity chromatography under a denaturating condition, and any other purification system may be used which is capable of purifying recombinant Sm29. First of all, the nickel column is balanced with a denaturation binding buffer, followed by the solution containing the recombinant protein. After washing the column with a binding buffer to eliminate contaminants, the recombinant protein is removed from the column with an elution buffer. The desalinization of the purified Sm29 is done in a gel filtration column, and any other desalinization method may be used which is capable of exchanging the elution buffer with a physiological buffer. To this end, the gel filtration column is balanced with a physiological buffer, followed by the purified protein. The separation of the salt contained in the recombinant purified protein occurs when it passes through the column, wherein the purified protein denaturating buffer is substituted with a physiological buffer.

1-29. (canceled)

30. A recombinant membrane protein Sm29 comprising the amino acid sequence of SEQ ID NO: 3.

31. A process for obtaining a recombinant protein Sm29, the process comprising:
(a) amplifying, by polymerase chain reaction, cDNA which corresponds to the Sm29 transcript, using specific primers, in an appropriate cDNA library (adult worm or schistosomulum);
(b) subcloning the amplified cDNA into ET21a or another expression vector which expresses the recombinant protein Sm29 and a C-terminal fusion of six histidines, in BL21(DE3) E. coli or another prokaryotic or eukaryotic expression system;
(c) inducing the expression vector which encodes the recombinant protein SM29 comprising the amino acid sequence from residue 40 to 169 of SEQ ID NO: 1, without N-terminal signal peptide or C-terminal transmembrane helix;
(d) collecting cells by centrifugation, re-suspending cells in a lysis buffer, and lysing cells by sonication;
(e) from the lysed cells, resuspending inclusion bodies containing recombinant protein in a denaturating buffer; and
(f) purifying the recombinant protein Sm29 in a six-histidine tag system based on nickel affinity chromatography under denaturating conditions or by another purification system which is capable of purifying recombinant protein Sm29.

32. A membrane protein Sm29 comprising the amino acid sequence of SEQ ID NO: 1.

33. The recombinant protein Sm29 of claim 30 or the membrane protein Sm29 of claim 32, wherein the protein has several HLA binding peptides selected from the group consisting of HLA DRB1*0101, HLA-DRB1*0301 (DR17), HLA-DRB1*0401 (DR4Dw4), HLA-DRB1*0701, HLA-DRB1*1101, and HLA-DRB1*1501 (DR2b).

34. A vaccine against schistosomiasis or fasciolosis comprising a recombinant Sm29 protein as defined in claim 30, a membrane protein Sm29 as defined in claim 32, or a salt thereof with a pharmacologically and physiologically acceptable carrier.

35. The vaccine of claim 34 further comprising adjuvant and/or cytokine.

36. Use of a recombinant protein Sm29 as defined in claim 30 or a membrane protein Sm29 as defined in claim 32, for inducing an effector response against schistosomiasis with production of antibodies and activation of cells of the immune system.

37. Use of a recombinant protein Sm29 as defined in claim 30 or a membrane protein Sm29 as defined in claim 32, for inducing high production of at least IgG isotype antibodies specific for Sm29 in a schistosomiasis patient or anti-Sm29 IgGl and IgG3 isotype antibodies in an individual resistant to schistosomiasis.

38. Use of a recombinant protein Sm29 as defined in claim 30 or a membrane protein Sm29 as defined in claim 32, for inducing immunological protection against schistosomiasis in recent phases of infection.

39. Use of a recombinant protein Sm29 as defined in claim 30 or a membrane protein Sm29 as defined in claim 32, for inducing high production of interleukin-10 to modulate an immune response.

40. Use of a recombinant protein Sm29 as defined in claim 30 or a membrane protein Sm29 as defined in claim 32, for suppressing a Th2 immune response and consequently reducing an allergic process.

41. Use of a recombinant protein Sm29 as defined in claim 30 or a membrane protein Sm29 as defined in claim 32, in a treatment method against allergens or treating an allergic disease.

42. Use of a recombinant protein Sm29 as defined in claim 30 or a membrane protein Sm29 as defined in claim 32, for generating a protective immunity against an infection caused by a helminth, including Schistosoma spp. or Fasciola spp.

43. Use of a recombinant protein Sm29 as defined in claim 30 or a membrane protein Sm29 as defined in claim 32, as a vaccine for inducing a seropositive antibody response against Schistosoma spp. in a mammal.

44. Use of a recombinant protein Sm29 as defined in claim 30 or a membrane protein Sm29 as defined in claim 32, as a vaccine for inducing a seropositive antibody response against Fasciola spp. in a mammal, especially bovine, caprine, or ovine.

45. A method of vaccination against schistosomiasis or fasciolosis comprising administration of a vaccine of claim 34 or 35 in one or more doses carried out by injection.

46. A pharmaceutical composition for modulating the immune system comprising a recombinant protein Sm29 as defined in claim 30, a membrane protein Sm29 as defined in claim 32, or a salt thereof with a pharmacologically and physiologically acceptable carrier.

47. An immunoenzymatic assay for diagnosing schistosomiasis comprising:
(a) adsorption of a recombinant protein Sm29 as defined in claim 30 or a membrane protein Sm29 as defined in claim 32, on a solid support;
(b) blocking of nonspecific binding sites on the Sm29-adsorbed support;
(c) binding control sera and test sera to the blocked support; and
(d) detecting anti-Sm29 antibody on the bound support.

48. An immunoenzymatic assay kit for diagnosing schistosomiasis comprising a recombinant protein Sm29 as defined in claim 30 or a membrane protein Sm29 as defined in claim 32, on a solid support.

49. The kit of claim 48, comprising a purified and desalted Sm29, test serum and control serum diluted in buffer containing TWEEN detergent, a conjugate having specificity for a target immunoglobulin which is anti-immunoglobulin conjugated to peroxidase, acetylcholinesterase, lactate dehydrogenase, β-galactosidase, glucose oxidase, or alkaline phosphatase, ortho-phenylenediamine substrate and sulfuric acid.


SEQUENCE LISTING (3 sequences)

SEQ ID NO: 1 - protein, 191 aa, Schistosoma mansoni
MKSGWEWIGIFLYIMVNILDKQRCHSVRCYVCDYCPIVTSVSISEENNCTSCSTAGYNYSIHRICVFKDGIPINFPNENRTQCNTDLCNGLTVDNTGKIPSVPIANPFRCYTCLNCTKSNQKVLSGCGACVTTRGSGIISKFCGTTCERLYIDDQISCCSTDLCNGMTKLSIHRHVIIVLFVCIGISKYIL

SEQ ID NO: 2 - protein, 165 aa, Schistosoma mansoni
VRCYVCDYCPIVTSVSISEENNCTSCSTAGYNYSIHRICVFKDGIPINFPNENRTQCNTDLCNGLTVDNTGKIPSVPIANPFRCYTCLNCTKSNQKVLSGCGACVTTRGSGIISKFCGTTCERLYIDDQISCCSTDLCNGMTKLSIHRHVIIVLFVCIGISKYIL

SEQ ID NO: 3 - protein, 165 aa, Schistosoma mansoni
MASMTGGQQMGRGSEFVRCYVCDYCPIVTSVSISEENNCTSCSTAGYNYSIHRICVFKDGIPINFPNENRTQCNTDLCNGLTVDNTGKIPSVPIANPFRCYTCLNCTKSNQKVLSGCGACVTTRGSGIISKFCGTTCERLYIDDQISCCSTDLCNGMTKHHHHHH